(19) United States
(12) Patent Application Publication
(10) Pub. No.: US 2023/0169840 A1
(43) Pub. Date: Jun. 1, 2023

(54) SYSTEM AND METHOD FOR MANAGING ACCESS TO AND OCCUPATION OF A LOCATION BY INDIVIDUALS BASED ON PHYSIOLOGICAL MEASUREMENT OF INDIVIDUALS

(71) Applicant: Minuteman Security Technologies, Inc, Andover, MA (US)

(72) Inventor: Joseph E. Lynch, Salem, NH (US)

(21) Appl. No.: 17/922,026

(22) PCT Filed: May 4, 2021
(86) PCT No.: PCT/US2021/030691

§ 371 Date: Oct. 28, 2022

Related U.S. Application Data
(60) Provisional application No. 63/022,090, filed on May 8, 2020.
(60) Provisional application No. 63/020,376, filed on May 5, 2020.

(51) Int. Cl. G08B 21/02; G07C 9/38

(52) U.S. Cl. CPC G08B 21/02; G07C 9/38

(57) ABSTRACT

A method and apparatus for managing access to, and occupation of, a location by one or more individuals, is provided. A location access control system is provided having data access to a maintained registry of one or more individuals allowed access to the location and configured to detect the presence of the one or more individuals at the location. Repeated physiological measurement information for one or more individuals detected as physically present at the location is received by the system. The repeated physiological measurement information is compared to a threshold level and when the physiological measurement information exceeds the threshold level, a notification is issued to the one or more individual associated with the physiological measurement information they must immediately vacate the location, or as a result of the automatic notification, be subject to further examination by security or medical staff.

### CROSS-REFERENCE TO RELATED APPLICATION(S)

This application claims priority to, and the benefit of, U.S. Provisional Application No. 63/020,376 filed May 5, 2020 and U.S. Provisional Application No. 63/022,090 filed May 8, 2020 for all subject matter contained in said applications. The disclosures of said provisional applications are hereby incorporated by reference in their entirety.

### FIELD OF THE INVENTION

The present invention relates to managing access to, and occupation of, a location. In particular, the present invention relates to managing access to, and occupation of, a location by individuals based on the physiological measurements of the individuals.

### BACKGROUND

Traditional locations management systems typically only make determinations as to whether an individual has access to the locations, typically this involves checking an identification. In some locations, where the health of the occupants is of concern, such as hospitals or other locations during a health crisis, additional screening may be performed that may include taking an individual's temperature. However, typically, an individual's temperature is only taken once, right before they enter the facility. Such screening cannot account for individuals whose temperature may fluctuate. For example, an individual's temperature may increase (indicating potential illness) after gaining access to the location. Similarly, an individual may have previously had a high temperature (again indicating illness) prior to coming to the location and taken a temperature-reducing medication to temporarily lower their temperature.

### SUMMARY

There is a need for an integrated, continuous human body physiological measurements monitoring system that could help predict illness and automatically report that condition to a person's workplace health & safety monitoring and notification system. The health & safety notification system could also have the ability to manage an infected person's access control to the workplace. There is additionally a need for managing access and occupation of a location that can track the physiological measurements of individuals having access to the location over time. The present invention is directed toward further solutions to address this need, in addition to having other desirable characteristics. Specifically, the present invention monitors the physiological measurements of individuals at the location and if an individual is determined to have a physiological measurement or a pattern of recent physiological measurement readings indicating potential illness, the individual is notified to vacate the location immediately. Thus, the present invention helps reduce the spread of illnesses at the location by minimizing the exposure of the potentially ill individual to other individuals at the location.

In accordance with an embodiment of the present invention, a method of managing access to, and occupation of, a location by one or more individuals, is provided. The method includes providing a building or location access control system having data access to a maintained registry of one or more individuals allowed access to the location and configured to detect the presence of the one or more individuals at the location; receiving repeated physiological measurement information for one or more individuals detected as physically present at the location; comparing the repeated physiological measurement information to a threshold level; and when the physiological measurement information exceeds the threshold level, automatically updating the location access control system and issuing a notification to the one or more individual associated with the physiological measurement information they must immediately vacate the location or as a result of the automatic notification, be subject to further examination by security or medical staff. To facilitate this functionality, the maintained registry includes contact information for the one or more individuals to facilitate the notification and the physiological measurement information is recorded and received automatically at a predetermined frequency.

In accordance with aspects of the present invention, the physiological measurement information is provided by a personal physiological measurements sensing device on each of the one or more individuals at the location. In some such aspects, the physiological measurement information is provided by a third-party service that collects information from personal physiological measurement sensing devices on individuals at the location. In other aspects, the physiological measurement information is received by one or more devices at the location configured to acquire physiological measurement information. In some aspects, the physiological measurement information comprises historical physiological measurement information for the individual. In certain aspects, receiving physiological measurement information involves sending a request for physiological measurement information for one or more individuals at the location, and receiving in response to the request, physiological measurement information for the one or more individuals.

In accordance with aspects of the present invention, the notification comprises a text message, email, or pop-up notification to one or more users.

In accordance with aspects of the present invention, the location comprises a workplace, school, or community gathering place.

In accordance with aspects of the present invention, the method further includes preventing an individual with physiological measurement information exceeding the threshold from entering the location.

In accordance with aspects of the present invention, the method further includes receiving location information data for the one or more individuals issued a notification.

In accordance with aspects of the present invention, the physiological measurement includes one or more of an individual's temperature, blood oxygen level, heart rate including resting heart rate, and respiratory rate.

In accordance with aspects of the present invention, the physiological measurement information is provided to a third-party cloud service that stores the information.

In accordance with aspects of the present invention, health data other than the physiological measurement information is provided to a third-party cloud service that stores the information.

In accordance with aspects of the present invention, a third-party service provides data storage and analysis of the physiological measurement information and generates a status indication relating to whether the individual is contagious

In accordance with embodiments of the present invention, a system for managing access to, and occupation of, a location by one or more individuals is provided. The system includes a registry of one or more individuals granted access to the location; one or more physiological measurement sensing devices that collect physiological measurement information on the one or more individuals; a location access control system managing access and occupation of the location having data access to the registry, the location access control system detecting the presence of one or more individuals at the location; and a monitoring system. The monitoring system receives repeated physiological measurement information associated with the one or more individuals who are detected as physically present at the location by the location access control system; compares the repeated physiological measurement information to a threshold level; and when the physiological measurement information exceeds the threshold level, automatically updates the location access control system, and utilizing the contact information, issues a notification to the one or more individual associated with the physiological measurement information they must immediately vacate the location. To facilitate this functionality, the registry includes contact information for the one or more individuals and the physiological measurement information is recorded and received automatically at a predetermined frequency.

In accordance with aspects of the present invention, the one or more physiological measurement sensing devices comprise a personal physiological measurement sensing device on each of the one or more individuals at the location. In some such aspects, the physiological measurement information is provided by a third-party service that collects information from personal physiological measurement sensing devices on individuals at the location. In other aspects, the physiological measurement sensing devices comprise one or more devices at the location configured to acquire physiological measurement information. In some aspects, the physiological measurement information comprises historical physiological measurement information for the individual.

In accordance with aspects of the present invention, the notification comprises a text message, email, or pop-up notification to one or more users.

In accordance with aspects of the present invention, the location comprises a workplace, school, or community gathering place.

In accordance with aspects of the present invention, the location access control system is further configured to prevent an individual with physiological measurement information exceeding the threshold from entering the location.

In accordance with aspects of the present invention, the monitoring system is further configured to receive location information data for the one or more individuals issued a notification.

In accordance with aspects of the present invention, the physiological measurement includes one or more of an individual's temperature, blood oxygen level, heart rate including resting heart rate, and respiratory rate.

In accordance with aspects of the present invention, the physiological measurement information is provided to a third-party cloud service that stores the information.

In accordance with aspects of the present invention, health data other than the physiological measurement information is provided to a third-party cloud service that stores the information.

In accordance with aspects of the present invention, a third-party service provides data storage and analysis of the physiological measurement information and generates a status indication relating to whether the individual is contagious

### BRIEF DESCRIPTION OF THE FIGURES

These and other characteristics of the present invention will be more fully understood by reference to the following detailed description in conjunction with the attached drawings, in which:

FIG. 1 is a diagrammatic illustration showing an environment in which a system and method for managing access to and occupation of a location by individuals based on physiological measurement of individuals is deployed;

FIG. 2 is a flow diagram depicting the method of FIG. 1; and

FIG. 3 is a diagrammatic illustration of a computing device and related hardware for use in implementation of the system of FIG. 1.

### DETAILED DESCRIPTION

An illustrative embodiment of the present invention relates to systems and methods for managing access to, and occupation of, a location by one or more individuals, based on repeated physiological measurement readings from the one or more individuals.

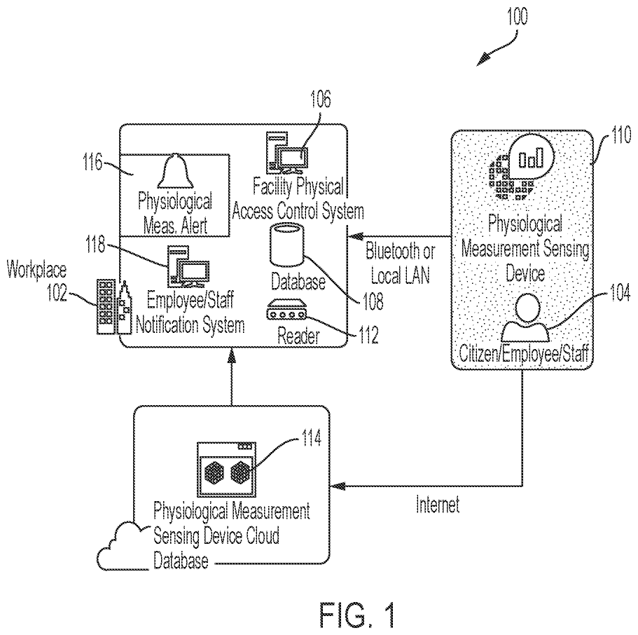

FIGS. 1 through FIG. 3 wherein like parts are designated by like reference numerals throughout, illustrate an example embodiment or embodiments of systems and methods for managing access to, and occupation of, a location by one or more individuals, based on repeated physiological measurement readings from the one or more individuals, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

FIG. 1 depicts an example deployment of a system 100 of the present invention at a location 102 in accordance with one embodiment. In this embodiment, the location 102 is a workplace and the one or more individuals 104 are the employees of the workplace. Other possible locations include, schools, universities, churches, community gathering places, checkpoint, venue, transportation hub, vehicle, or location having a form of controlled access.

The system 100 includes a location access control system 106. The location access control system 106 has data access to a maintained registry 108 of one or more individuals 104 allowed access to the location 102. The registry 108 includes contact information for the one or more individuals 104. One or more physiological measurement sensing devices 110 collect physiological measurement information from the one or more individuals 104. As utilized herein, physiological measurement(s) refers to an individual's temperature, blood oxygen level, heart rate including resting heart rate, respiratory rate, and/or the like. The physiological measurements leveraged by the present invention are those measurements that can be secured using the system described herein, and which can be utilized as data relevant to ascertaining whether an individual is experiencing an illness or is cable of spreading an illness. A monitoring system 116 receives the physiological measurement information for one or more individuals 104 detected by the location access control system 106 as being physically present at the location 102 and compares the physiological measurement information to a threshold level. When the physiological measurement information exceeds the threshold level, the monitoring system 116 automatically updates the location access control system 106 and, using the contact information, issues a notification to the one or more individuals 104 associated with the physiological measurement information they must immediately vacate the location 102, or as a result of the automatic notification, be subject to further examination by security or medical staff.

As utilized herein, the “location” being accessed or occupied by the individual can be any number of locations, including a workplace, school, community gathering place, building, checkpoint, venue, transportation hub, vehicle, or location having a form of controlled access.

The location access control system 106 is configured to detect the presence of one or more individuals 104 at the location. The location access control system 106 can include any of the functionality typically implemented to controls access to a location 102, such as a workplace including locked doors or security checkpoints that only provide access for authorized personnel. In certain embodiments, this is performed in conjunction with sensors, such as ID badge readers, biometric scanners, or cameras, or other equipment such as keypads or visitor check-in systems. Other implementations or configurations for detecting the presence of individuals 104 will be apparent to one skilled in the art given the benefit of this disclosure.

In some embodiment, the location access control system is further configured to prevent an individual 104 access to the location 102 if the individual is determined to have a physiological measurement above a threshold value.

The maintained registry 108 contains information regarding the one more individuals 104 that are allowed access to the location 102. This information can include, but is not limited to: name, member or employee registration data, security access or authorization privileges for location, physiological measurement monitoring authorizations or policies for individuals, and contact information. Other possible information to be included in the registry 108. The contact information is what is used to provide the notification. This contact information may also include the preferred method of notification. In certain embodiments, an individual's inclusion in the registry 108 also serves as authorization to have physiological measurement information about the one or more individuals 104 provided to the system 100 as well as the having policy or procedures for receiving the physiological measurement information for the one or more individual 104.

The physiological measurement information for the one or more individuals is collected by one or more physiological measurement sensing devices. In the embodiment of FIG. 1, the one or more physiological measurement sensing devices include a personal physiological measurement sensing device 110 on each of the individuals 104 being monitored. Examples, of personal physiological measurement sensing devices 110 include physiological data tracking devices that are wearable devices, worn on the person, such as watches, rings, headbands, pendants, bracelets, patches, badges, or the like that are configured to collect information, such as physiological measurements, repeatedly and periodically from the individual who wears the device. Some example devices can include but are not limited to the Oura® ring, Garmin® smartwatches, Apple® watch, Samsung® smartwatch, and the like. In many instances these devices 110 are designed to be used in conjunction with an application running on a smart device or computer, including mobile devices, that collects, stores, categorizes, organizes, and presents the information. In some instances, the application or the device 110 itself provides the collected information to a third-party monitoring service 114, such as a website or cloud service, that tracks and keeps a record of the collected information. Likewise, the cloud service can include data storage of information collected, such as the physiological measurements, but also can include data storage of information separately collected and entered, such as test results such as recent test results for presence of a viral or contagious illness. Test results could include the presence of a particular viral illness anti-body found in an individual. The cloud services can be provided as a third-party service, where both the individual whose physiological measurements are collected, and businesses or other locations can be subscribers to the third-party cloud service. Likewise, the third-party cloud service can serve as a repository for “contagious” vs. “healthy” individuals at any one point in time, indicating such status upon request. The status indicator can be based on the third-party system applying criteria or algorithms analyzing the collected physiological measurement information according to any number of known processes for diagnosis and prognosis as would be understood by those of skill in the art.

In certain embodiments, the one or more physiological measurement sensing devices can include one or more physiological measurement information collection devices 112 provided at and around the location 102 to obtain physiological measurement information from the one or more individuals 104 that are present at the location 102. In some embodiments, these physiological measurement information collection devices 112 are in the form of readers configured to received physiological measurement information from the personal physiological measurement sensing devices 110 on each individual 104. The information may be provided by the device 110 or application used in conjunction with the device 110. In some such embodiments, the physiological measurement information collection device 112 sends a request for information and receives the physiological measurement information directly from the personal device 110 or application in response. The information, including the request for information, can be transmitted using any number of data transfer protocols including wireless protocols such as Wi-Fi, Bluetooth®, and near field communication (NFC) communications.

In other embodiments, the one or more physiological measurement information collection devices 112 can be configured to take or otherwise read an individual's physiological measurements. In such embodiments, the physiological measurement information collection devices 112 may include an infrared camera or other sensor that can detect the physiological measurement(s) of the one or more individuals 104 physically present at the location 102.

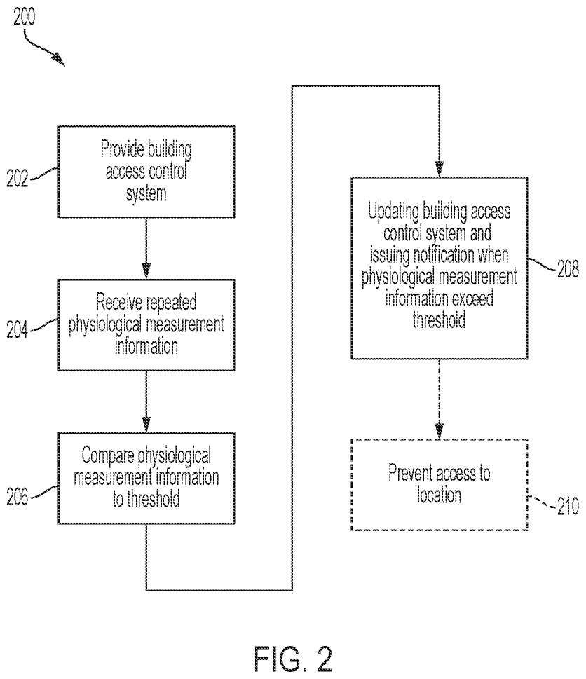

In operation, a monitoring system 116 is in communication with the location access control system 106 and the registry 108. The monitoring system 116 is configured to receive repeated physiological measurement information associated with the one or more individuals 104 in the maintained registry 108 who are detected as physically present at the location 102 by the location access control system 106. The monitoring system 116 compares the repeated physiological measurement information to a threshold level. When the physiological measurement information exceeds the physiological measurement threshold, the monitoring system then issues a notification to the one or more individuals associated with the physiological measurement information that they must immediately vacate the location. A flow diagram 200 for implementing this a methodology in accordance with one embodiment can be seen in FIG. 2.

If the first step 202 of the methodology of FIG. 2, a location access control system 106 as described herein is provided (Step 202). As discussed above, the location access control system 106 has data access to a maintained registry 108 and is configured to detect the presence of one or more individuals 104 at the location 102.

The repeated physiological measurement information is received by the monitoring system 116 from the physiological measurement sensing devices via the physiological measurement information collection devices 112 or communication with a service 114 that collects physiological measurement information for the individuals being monitored (Step 204). In some such embodiments, the receiving the physiological measurement information involves sending a request for information and receiving the physiological measurement information from the physiological measurement information collection device 112 or service 114 in response. The information, including the request for information, can be transmitted using any number of data transfer protocols including wired and/or wireless protocols. In some such embodiments, the monitoring system 116, service 114, and personal physiological measurement sensing device 110 (and associate application) are registered with each other as being authorized to access, provide, and receive physiological measurement information for the one or more individuals 104. In some further embodiments, entry of data in the registry 108 includes registrations and permissions to obtain and use physiological measurement information.

The physiological measurement information is repeated in that it is received on a periodic basis at a predetermined frequency through the time period individual is physically present at the location. The frequency can vary based on the needs and/or requirements of the system 100. The repeated physiological measurement information may be received as frequently as it is obtained by the physiological measurement sensing device or service 114, or other at some other predetermined frequency. In some embodiments, the registry 108 may include specification or requirements for the frequency or amount of repeated physiological measurement information received and recorded by the monitoring system 116.

In some embodiments, the received repeated physiological measurement information may include historical physiological measurement information including an individual's physiological measurement(s) prior to their arrival or detection at the location. Such historical physiological measurement(s) information may be recorded and stored by the personal physiological measurement sensing device 110, or the application and/or service used in conjunction with the personal physiological measurement sensing device 110. Similarly, the monitoring system may also store historical physiological measurement data for the one or more individuals.

In the next step of FIG. 2, the received repeated physiological measurement information is compared to a threshold level (Step 206). The threshold level for the repeated physiological measurement information can be a standardized threshold for all the individuals 104 being monitored at the location 102 (e.g. 100 degrees Fahrenheit/38 degrees Celsius) or be unique for each individual 104 being monitored at the location 102. For example, some individuals, may run a hotter or colder temperature than average, where a standardized threshold value would not accurately reflect their current condition. In some such embodiments, such unique temperature information for an individual could be included in the registry 108 or store by the monitoring system 116.

When the received repeated physiological measurement information exceeds the threshold, the location access control system 106 is updated and a notification is issued to the one or more individuals 104 associated with the temperature information that they must vacate the location 102 (step 208). The notification is issued using the contact information included in the maintained registry 108. The notification can be provided by voice message, text, email, instant message, pop-up notification, or other suitable mechanism. In certain embodiments, the registry 108 may include specification for notification means for the each of the one or more individuals 104. In some embodiments, other individuals, such as faculty, owners or management of the location 102, may be notified of the individual having a physiological measurement exceeding a threshold level.

In some instances, the monitoring system 116 may provide a monitoring portal, such a through a website or application, where status updates including notifications are provided. Such a portal can also be used to maintain the registry 108.

In some embodiments, a separate or third-party notification system 118 may be used to provide the notification. For example, some locations 102, such as schools or business, may already have a notification system implemented for providing notifications regarding closures, power failures, fire alarms, security alerts, or the like. In such instances, the monitoring system 116 can be in communication or otherwise integrated with the notification system 118 to make use of the notification system 118 for issuing notifications.

In some embodiments, in addition to issuing a notification (step 208), the location control access system may take the further step of preventing the one or more individuals 104 having physiological measurement information exceeding the threshold from the accessing the location 102 (step 210). For example, the doors of the location, that would normally be unlocked for the individual would not be unlocked.

In some embodiments, once an individual 104 is issued a notification, the monitoring system 116 may receive or otherwise obtain location information data about the individual in order to trace potential exposure to the potentially ill individual. In some such embodiments, the personal physiological measurement sensing device 110 (or associated application or smart device running application) may further be capable of providing Global Positioning System (GPS) coordinates, WiFi location coordinates, or other location tracking protocols or services, for the individual 104. In some such embodiments, the location information data may include historical location information. Permissions and/or protocols for providing such location information data can be included in the registry 108.

It should be understood that while FIG. 1 depicts an implementation having the location access control system 106, registry 108, and monitoring system 116 as separate element provided at the location 102. The individual components do not have to reside at the physical location. Indeed, some all or some of the location access control system 106, registry 108, and monitoring system 116 can be implemented remotely, or distributed across multiple systems, or device. Similarly, the functionality of the various components can be combined together without departing from the spirit of the invention.

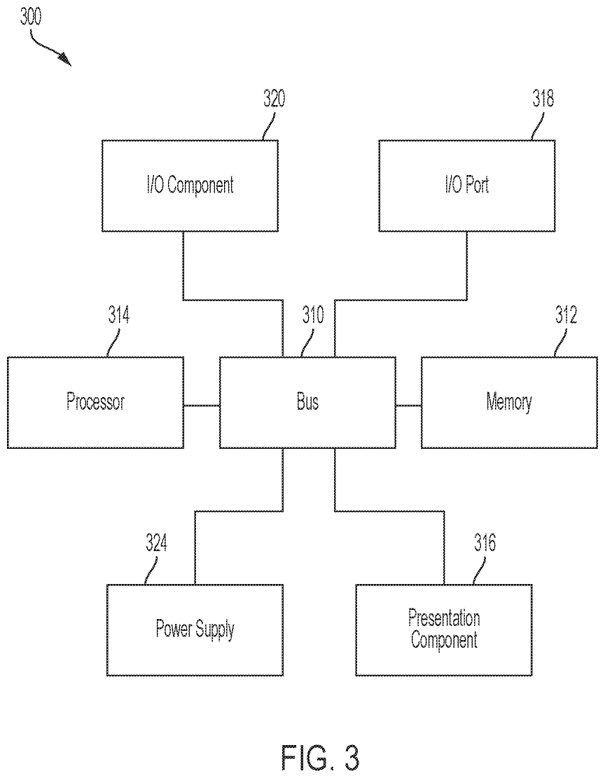

FIG. 3 depicts an example electronic, computer, or computing device 300 that can be used to implement one or more aspects of the present invention, including the location access control system 106, the physiological measurement sensing devices, the monitoring system 116, monitoring services 114, notification system 118, as well as other functionality described herein. The functionality and hardware of such computing device 300 may be implemented in any of the electronic hardware systems or subsystems described herein as involving or using a “computer” or “computing device” or the like, or related hardware for providing all or part of the described functionality, provided as a separate device or integrated into a system or subsystem described herein, as would be appreciated and understood by those of skill in the art. The terms “computer”, “computing device”, and the like utilized herein are intended to mean a processor at its most basic form, on up to more complex computing systems, including servers and cloud-based systems, in accordance with conventional meanings of such terms. However, for purpose of completeness, example components and related accessories that are intended to be encompassed by the use of the terms “computer”, “computing device”, “processor”, and the like will be provided below in example nonlimiting form.

The computing device 300 is merely an illustrative example of a suitable computing environment and in no way limits the scope of the present invention. An “autonomous access controller”, “electronic device”, “remote device,” or “personal electronic device” as represented in figures and description herein, can include a “workstation,” a “server,” a “laptop,” a “desktop,” a “hand-held device,” a “mobile device,” a “tablet computer,” a “processor,” or other computing devices, as would be understood by those of skill in the art. Given that the computing device 300 is depicted for illustrative purposes, embodiments of the present invention may utilize any number of computing devices 300 in any number of different ways to implement a single embodiment of the present invention. Accordingly, embodiments of the present invention are not limited to a single computing device 300, as would be appreciated by one with skill in the art, nor are they limited to a single type of implementation or configuration of the example computing device 300.

The computing device 300 can include a bus 310 that can be coupled to one or more of the following illustrative components, directly or indirectly: a memory 312, one or more processors 314, one or more presentation components 316, input/output ports 318, input/output components 320, and a power supply 324. One of skill in the art will appreciate that the bus 310 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, the figures herein are merely illustrative of an exemplary computing device 300 that can be used to implement one or more embodiments of the present invention, and in no way limits the invention.

The computing device 300 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 300.

The memory 312 can include computer-storage media in the form of volatile and/or nonvolatile memory. The memory 312 may be removable, non-removable, or any combination thereof. Exemplary hardware devices are devices such as hard drives, solid-state memory, optical-disc drives, and the like. The computing device 300 can include one or more processors 314 that read data from components such as the memory 312, the various I/O components 320, etc. Presentation component(s) 316 present data indications to a user or other device. Exemplary presentation components include a display device, speaker, printing component, vibrating component, etc.

The I/O ports 318 can enable the computing or electronic device 300 to be logically coupled to other devices, such as I/O components 320. Some of the I/O components 320 can be built into the computing device 300. Examples of such I/O components 320 include a sensor (including but not limited to: a camera, optical scanner, RFID scanner, or the like), keypad, touchpad, joystick, recording or storage device, game pad, satellite dish, scanner, printer, wireless device, networking device, and the like, as appropriate.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A method of managing access to, and occupation of, a location by one or more individuals, the method comprising:
providing a location access control system managing access and occupation of the location having data access to a maintained registry of one or more individuals allowed access to the location, the registry including contact information for the one or more individuals, the location access control system configured to detect a presence of the one or more individuals at the location;
receiving repeated physiological measurement information associated with the one or more individuals who are detected as physically present at the location by the location access control system, the physiological measurement information recorded and received automatically at a predetermined frequency;
comparing the repeated physiological measurement information to a threshold level; and
when the physiological measurement information exceeds the threshold level, automatically updating the location access control system, and utilizing the contact information, issuing a notification to the one or more individual associated with the physiological measurement information they must immediately vacate the location.

2. The method of claim 1, wherein the physiological measurement information is provided by a personal physiological measurement sensing device on each of the one or more individuals at the location.

3. The method of claim 1, wherein the physiological measurement information is provided by a third-party service that collects information from personal physiological measurement sensing devices on individuals at the location.

4. The method of claim 1, wherein receiving physiological measurement information comprises:
sending a request for physiological measurement information for one or more individuals at the location; and
receiving in response to the request, physiological measurement information for the one or more individuals.

5. The method of claim 1, wherein the notification comprises a text message, email, or pop-up notification to one or more users.

6. The method of claim 1, wherein the location comprises a workplace, school, community gathering place, location, checkpoint, venue, transportation hub, vehicle, or location having a form of controlled access.

7. The method of claim 1, wherein the physiological measurement information is received by one or more devices at the location configured to acquire physiological measurement information.

8. The method of claim 1, wherein the physiological measurement information comprises historical physiological measurement information for the individual.

9. The method of claim 1, further comprising preventing an individual with physiological measurement information exceeding the threshold from entering the location.

10. The method of claim 1, further comprising receiving location information data for the one or more individuals issued a notification.

11. The method of claim 1, wherein the physiological measurement comprises one or more of an individual's temperature, blood oxygen level, heart rate including resting heart rate, and respiratory rate.

12. The method of claim 1, wherein the physiological measurement information is provided to a third-party cloud service that stores the information.

13. The method of claim 1, wherein health data other than the physiological measurement information is provided to a third-party cloud service that stores the information.

14. The method of claim 1, wherein a third-party service provides data storage and analysis of the physiological measurement information and generates a status indication relating to whether the individual is contagious.

15. A system of managing access to, and occupation of, a location by one or more individuals, the system comprising:
a registry of one or more individuals granted access to the location, the registry including contact information for the one or more individuals;
one or more physiological measurement sensing devices that collects physiological measurement information on the one or more individuals;
a location access control system managing access and occupation of the location having data access to the registry, wherein the location access control system detects a presence of one or more individuals at the location; and
a monitoring system, wherein the monitoring system receives repeated physiological measurement information associated with the one or more individuals who are detected as physically present at the location by the location access control system, the physiological measurement information recorded and received automatically at a predetermined frequency, compares the repeated physiological measurement information to a threshold level, and when the physiological measurement information exceeds the threshold level, automatically updates the location access control system, and utilizing the contact information, issues a notification to the one or more individual associated with the physiological measurement information they must immediately vacate the location.

16. The system of claim 15, wherein the one or more physiological measurement sensing devices comprise a personal physiological measurement sensing device on each of the one or more individuals at the location.

17. The system of claim 15, wherein the physiological measurement information is provided by a third-party service that collects information from personal physiological measurement sensing devices on individuals at the location.

18. The system of claim 15, wherein the notification comprises a text message, email, or pop-up notification to one or more users.

19. The system of claim 15, wherein the location comprises a workplace, school, or community gathering place.

20. The system of claim 15, wherein the physiological measurement sensing devices comprise one or more devices at the location configured to acquire physiological measurement information.

21. The system of claim 15, wherein the physiological measurement information comprises historical physiological measurement information for the individual.

22. The system of claim 15, wherein the location access control system is further configured to prevent an individual with physiological measurement information exceeding the threshold from entering the location.

23. The system of claim 15, wherein the monitoring system receives location information for the one or more individuals issued a notification.

24. The system of claim 15, wherein the physiological measurement comprises one or more of an individual's temperature, blood oxygen level, heart rate including resting heart rate, and respiratory rate.

25. The system of claim 15, wherein the physiological measurement information is provided to a third-party cloud service that stores the information.

26. The system of claim 15, wherein health data other than the physiological measurement information is provided to a third-party cloud service that stores the information.

27. The system of claim 15, wherein a third-party service provides data storage and analysis of the physiological measurement information and generates a status indication relating to whether the individual is contagious.
